(19) United States
(12) Patent Application Publication
(10) Pub. No.: US 2025/0034067 A1
(43) Pub. Date: Jan. 30, 2025

(54) PURIFICATION PROCESS OF ORGANIC SOLVENT

(71) Applicant: DONGWOO FINE-CHEM CO., LTD., Iksan-si (KR)

(72) Inventors: Hwan-Min KIM, Iksan-si (KR); Dong-Seok SEO, Iksan-si (KR); Uk LEE, Iksan-si (KR)

(73) Assignee: DONGWOO FINE-CHEM CO., LTD., Iksan-si (KR)

(21) Appl. No.: 18/784,246

(22) Filed: Jul. 25, 2024

(30) Foreign Application Priority Data
Jul. 27, 2023 (KR) 10-2023-0098236

(51) Int. Cl. C07C 29/80; B01D 3/14

(52) U.S. Cl. CPC C07C 29/80; B01D 3/143

(57) ABSTRACT

The present disclosure relates to a purification process of an organic solvent, the process including: (a) performing distillation by supplying an organic solvent to a distillation column; and (b) discharging a portion of the organic solvent including impurities through a side discharge stream connected to a lower part of the distillation column, wherein the discharging of (b) discharges a portion of the organic solvent including impurities at a flow rate of 0.5% to 10% with respect to an input flow rate of the organic solvent supplied to the distillation column in (a).

### TECHNICAL FIELD

The present disclosure relates to a purification process of an organic solvent.

### BACKGROUND ART

Organic solvents used as process solutions for various purposes such as cleaning solutions in a semiconductor process are managed to have high quality by controlling the total amount of impurities, and an increase in the content of the impurities may lead to substrate contamination, process failure, wafer defects and the like. Specifically, when an organic solvent includes the impurities, errors occur during pattern formation, or adhesion of a deposited thin film is inhibited. In addition, there is a possibility that residual organic contaminants may react with a silicon substrate during a thermal process to form compounds such as silicon carbide. This may cause damage to electrical performance of a semiconductor circuit, which may ultimately affect stability of performance of a device including the same and overall productivity.

In addition, removed organic impurities may be regenerated depending on management conditions of the organic solvent, one of which is acetone. Generation of acetone reduces purity of a purified solvent, and reduces cleaning power, which leads to a decrease in the process yield. Furthermore, proper function of a solvent may not be performed in a semiconductor process.

Korean Patent Application Laid-Open No. 10-2021-0070275 discloses a method for preparing isopropyl alcohol by directly hydrating propylene with water, the method including a distillation process of distilling crude isopropyl alcohol, and a filtration process of filtering the isopropyl alcohol obtained in the distillation process with a filter having an ion-exchange group.

However, such a related art may have problems in that organic impurities such as acetone and/or metal impurities are not sufficiently removed and remain even after purification, and impurities may be regenerated during a storage process of a purified organic solvent

### PRIOR ART DOCUMENTS

### Patent Documents

(Patent Document 1] Korean Patent Application Laid-Open No. 10-2021-0070275

### DISCLOSURE

### Technical Problem

The present disclosure has been made in view of the above, and is directed to providing a purification process of an organic solvent capable of obtaining a high-purity organic solvent by including a step of continuously discharging impurities having relatively high boiling points at a certain flow rate together with a portion of an organic solvent through a side discharge stream connected to a lower part of a distillation column, thereby efficiently removing organic impurities such as N-propyl alcohol and/or metal impurities.

In addition, the present disclosure is directed to suppressing regeneration of organic impurities such as acetone even during long-term storage by controlling a storage temperature of a purified organic solvent.

In addition, the present disclosure is directed to providing a high-purity organic solvent obtained by continuously discharging impurities having relatively high boiling points at a certain flow rate through a side discharge stream.

However, objects to be addressed by the present disclosure are not limited to the objects mentioned above, and other objects not mentioned will be clearly appreciated by those skilled in the art from the following description.

### Technical Solution

One embodiment of the present disclosure relates to a purification process of an organic solvent, the process including: (a) performing distillation by supplying an organic solvent to a distillation column; and (b) discharging a portion of the organic solvent including impurities through a side discharge stream connected to a lower part of the distillation column, wherein the discharging of (b) discharges a portion of the organic solvent including impurities at a flow rate of 0.5% to 10% with respect to an input flow rate of the organic solvent supplied to the distillation column in (a).

In the present disclosure, the organic solvent may be an alcohol-based organic solvent.

In the present disclosure, the organic solvent may be one or more selected from among isopropyl alcohol, propyl alcohol, butyl alcohol and hexyl alcohol.

In the present disclosure, the impurity may be a substance having the same or higher boiling point compared to the organic solvent.

In the present disclosure, the impurity may include one or more selected from among N-propyl alcohol, 2-pentanone, 4-methyl-2-pentanone, 2-butanol, isopropyl acetate, nonanaldehyde, decanaldehyde, T-amyl alcohol, diacetone alcohol and metal impurities.

In the present disclosure, the discharging of (b) may discharge at a flow rate of 1.0% to 10% with respect to an input flow rate of the organic solvent supplied to the distillation column in (a).

In the present disclosure, the discharging of (b) may be performed as a continuous process.

In the present disclosure, the purification process of an organic solvent may be for reducing a content of at least one of iron (Fe), chromium (Cr), nickel (Ni) and copper (Cu) included in the organic solvent to 30% or less.

In the present disclosure, the purification process of an organic solvent may further include storing the purified organic solvent at a temperature of lower than 35° C.

In the present disclosure, the purification process of an organic solvent may further include storing the purified organic solvent at a temperature of 30° C. or lower.

In the present disclosure, the purification process of an organic solvent may further include at least one of removing moisture included in the organic solvent prior to (a); re-vaporizing the liquefied impurities discharged through the lower part of the distillation column after (a) and prior to (b); liquefying the distilled organic solvent through a condenser after (a); and refluxing the organic solvent distilled in the distillation column after (a).

One embodiment of the present disclosure relates to an organic solvent in which a content of at least one of N-propyl alcohol, 2-pentanone, 4-methyl-2-pentanone, 2-butanol, isopropyl acetate, nonanaldehyde, decanaldehyde, T-amyl alcohol and diacetone alcohol is 2 ppm or less in concentration, and a content of at least one of iron (Fe), chromium (Cr), nickel (Ni) and copper (Cu) is 5 ppt or less in concentration, and includes an organic solvent purified according to the organic solvent purification process described above.

### Advantageous Effects

According to an organic solvent purification process according to the present disclosure, purity of a purified organic solvent can be further improved by continuously discharging an organic solvent including impurities through a side discharge stream at a flow rate of 0.5% to 10%, more preferably 1.0% to 10%. Specifically, according to the present disclosure, organic impurities such as N-propyl alcohol and/or metal impurities included in an organic solvent can be effectively controlled.

In addition, according to an organic solvent purification process according to the present disclosure, regeneration of organic impurities such as acetone can be suppressed by storing a purified organic solvent at a temperature of lower than 35° C., preferably at a temperature of 30° C. or lower, and a purified organic solvent with reliability can be provided.

### BRIEF DESCRIPTION OF DRAWINGS

FIG. 1 illustrates a process diagram according to a purification process of an organic solvent of one embodiment of the present disclosure.

### BEST MODE

The present disclosure is for providing a purification process of an organic solvent, and the process includes: (a) performing distillation by supplying an organic solvent to a distillation column; and (b) discharging a portion of the organic solvent including impurities through a side discharge stream connected to a lower part of the distillation column, wherein the discharging of (b) discharges a portion of the organic solvent at a flow rate of 0.5% to 10% with respect to an input flow rate of the organic solvent supplied to the distillation column in (a), thereby relating to a purified organic solvent with high purity. In addition, the scope of the present disclosure includes an organic solvent purified according to the organic solvent purification process, and a storage process of the organic solvent.

### <Purification Process of Organic Solvent>

The organic solvent purification process of the present disclosure includes a step of continuously discharging a portion of an organic solvent including impurities in the distillation step at a specific flow rate. Specifically, by discharging a certain proportion of raw material, which includes impurities, collected at the lower part of the column, concentration of a substance falling to the lower part of the distillation column is prevented through continuous reflux in the distillation column, thereby improving purity of a final purified organic solvent. In the present disclosure, the raw material may mean an organic solvent before purification.

More specifically, the organic solvent purification process of the present disclosure includes: (a) performing distillation by supplying an organic solvent to a distillation column; and (b) discharging a portion of the organic solvent including impurities through a side discharge stream connected to a lower part of the distillation column, wherein the discharging of (b) may discharge a portion of the organic solvent at a flow rate of 0.5% to 10% with respect to an input flow rate of the organic solvent supplied to the distillation column in (a).

According to the organic solvent purification process of the present disclosure, organic impurities and/or metal impurities may be efficiently reduced.

Examples of the organic solvent may include methanol, ethanol, 1-propanol, isopropyl alcohol (isopropanol), n-propanol, 2-methyl-1-propanol, n-butanol, 2-butanol, tert-butanol, 1-pentanol, 2-pentanol, 3-pentanol, n-hexanol, cyclohexanol, 2-methyl-2-butanol, 3-methyl-2-butanol, 2-methyl-1-butanol, 3-methyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 2-ethyl-1-butanol, 2,2-dimethyl-3-pentanol, 2,3-dimethyl-3-pentanol, 2,4-dimethyl-3-pentanol, 4,4-dimethyl-2-pentanol, 3-ethyl-3-heptanol, 1-heptanol, 2-heptanol, 3-heptanol, 2-methyl-2-hexanol, 2-methyl-3-hexanol, 5-methyl-1-hexanol, 5-methyl-2-hexanol, 2-ethyl-1-hexanol, methylcyclohexanol, trimethylcyclohexanol, 4-methyl-3-heptanol, 6-methyl-2-heptanol, 1-octanol, 2-octanol, 3-octanol, 2-propyl-1-pentanol, 2,6-dimethyl-4-heptanol, 2-nonanol, 3,7-dimethyl-3-octanol, ethylene glycol, propylene glycol, diethyl ether, dipropyl ether, diisopropyl ether, butyl methyl ether, butyl ethyl ether, butyl propyl ether, dibutyl ether, diisobutyl ether, tert-butyl methyl ether, tert-butyl ethyl ether, tert-butyl propyl ether, di-tert-butyl ether, dipentyl ether, diisoamyl ether, cyclopentyl methyl ether, cyclohexyl methyl ether, bromomethyl methyl ether, α,α-dichloromethyl methyl ether, chloromethyl ethyl ether, 2-chloroethyl methyl ether, 2-bromoethyl methyl ether, 2,2-dichloroethyl methyl ether, 2-chloroethyl ethyl ether, 2-bromoethyl ethyl ether, (+)-1,2-dichloroethyl ethyl ether, 2,2,2-trifluoroethyl ether, ethyl vinyl ether, butyl vinyl ether, allyl ethyl ether, allyl propyl ether, allyl butyl ether, diallyl ether, 2-methoxypropene, ethyl-1-propenyl ether, cis-1-bromo-2-ethoxyethylene, 2-chloroethyl vinyl ether, allyl-1,1,2,2-tetrafluoroethyl ether, octane, isooctane, nonane, decane, methylcyclohexane, decalin, xylene, ethylbenzene, diethylbenzene, cumeme, sec-butylbenzene, cymene, dipentene, methyl pyruvate, monomethyl ether, propylene glycol monomethyl ether, propylene glycol monoethyl ether, propylene glycol monopropyl ether, propylene glycol monomethyl ether acetate, ethyl lactate, methyl methoxypropionate, cyclopentanone, cyclohexanone, n-butyl acetate, γ-butyrolactone, diisoamyl ether, isoamyl acetate, chloroform, dichloromethane, 1,4-dioxane, hexyl alcohol, 2-heptanone, isoamyl acetate, propylene carbonate, tetrahydrofuran and the like. In one embodiment of the present disclosure, as an alcohol-based organic solvent, one or more selected from among isopropyl alcohol, propyl alcohol, butyl alcohol and hexyl alcohol are more preferred, and isopropyl alcohol is most suitable for the present disclosure.

Hereinafter, preferred embodiments of the present disclosure will be described in detail with reference to drawings. However, these embodiments are presented only as an example in order to more specifically describe the present disclosure, and it will be obvious to those skilled in the art that the scope of the present disclosure is not limited by these embodiments.

The terms ‘include (comprise)’ and/or ‘including (comprising)’ used in the present specification are used in a sense of not excluding the presence or addition of one or more other constituents, steps, operations and/or constituents in addition to the mentioned constituents, steps, operations and/or the constituents.

The term ‘purification’ used in the present specification means removing impurities to purify a substance and does not mean removing 100% of impurities only, and may mean a flow in which a substance included in the flow passing through a dehydration or purification means is included in an amount of 70% by weight or greater, preferably 90% by weight or greater, and more preferably 95% by weight or greater or 99% by weight or greater. In the present disclosure, a ‘purified organic solvent’ may mean an organic solvent in a state in which organic impurities and/or metal impurities are removed, and may be at a level of 99.999% or greater.

The purification process of an organic solvent according to one embodiment of the present disclosure includes (a) performing distillation by supplying an organic solvent to a distillation column 120.

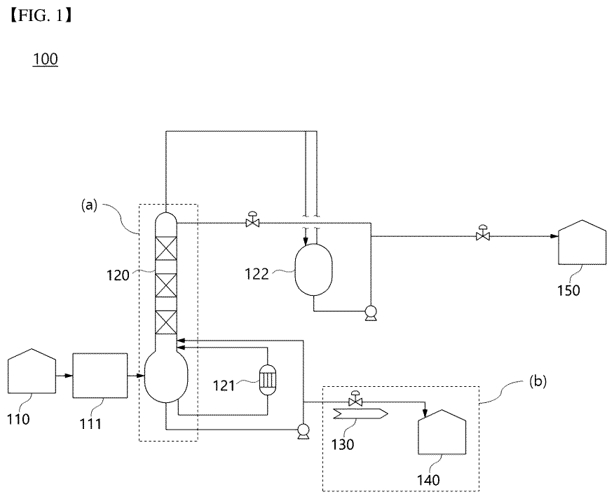

FIG. 1 illustrates a process diagram of a purification process of an organic solvent according to one embodiment of the present disclosure, and a purification tower 100 of the present disclosure may include at least one of a raw material tank 110, a dry bed unit 111, a distillation column 120, a reboiler 121, a reflux tank 122, a side discharge stream 130, an impurity discharge tank 140 and/or a product tank 150, and preferably includes all of these.

The purification process of an organic solvent according to one embodiment of the present disclosure may include, prior to the step of (a) performing distillation by supplying an organic solvent to a distillation column 120, a step of introducing an organic solvent raw material through a raw material tank 110 as a first step. The step of introducing an organic solvent raw material through the raw material tank 110 is not particularly limited, however, the organic solvent raw material may be introduced at a flow rate of 500 kg/hr to 6,500 kg/hr.

In the present disclosure, the purification process may further include, prior to the step (a) and after the step of introducing an organic solvent raw material through a raw material tank 110, a step of removing moisture included in the organic solvent. For the organic solvent raw material introduced through the raw material tank 110, moisture included in the organic solvent may be removed first in a dry bed unit 111 before being supplied to the distillation column 120 in order to increase purification efficiency. Herein, as the method of removing moisture, that is, the dehydration process, methods known in the art may be used without limit. Examples thereof may include an azeotropic distillation method, an extractive distillation method and a molecular sieve dehydration method, but are limited thereto. When the molecular sieve dehydration method is used in one embodiment of the present disclosure, zeolite is preferably used.

The organic solvent from which moisture is removed in the dry bed unit 111 is introduced into the distillation column 120, and the step (a) is performed. The step (a) may be a step of sorting impurities using a difference in the boiling points. A side discharge stream 130 is connected to the distillation column 120 of the present disclosure, and specifically, may be connected to a lower part of the distillation column 120 as illustrated in FIG. 1. In this case, organic impurities, metal impurities and/or the like having relatively high boiling points may be discharged to an impurity discharge tank 140 together with a portion of the organic solvent. In addition, when the discharge stream is connected to the upper part, organic impurities having relatively low boiling points and the like may be separated from the raw material.

In one embodiment of the present disclosure, the impurity may be a substance having the same or higher boiling point compared to the organic solvent, and includes organic impurities and metal impurities.

The organic impurities are the largest source of contamination of residues in a semiconductor device manufacturing process, and, by having hydrophobic surface properties, the organic impurities may significantly reduce cleaning effects, may cause micro-masking effects during the process, and may inhibit attachment of a deposited thin film when remaining without being removed.

The representative examples of metal impurities include iron (Fe), chromium (Cr), nickel (Ni), copper (Cu) and/or the like, and the metal impurities may contaminate a substrate even at a level of about 10¹¹ atoms/cm² to 10¹³ atoms/cm². A silicon substrate and the metal ions may directly bond through charge exchange, or the metal impurities may be included when forming an oxide film on a substrate surface to cause contamination. One embodiment of the present disclosure may be for reducing the content of at least one of iron (Fe), chromium (Cr), nickel (Ni) and copper (Cu) included in the organic solvent to 30% or less with respect to the raw material. Preferably, iron (Fe), chromium (Cr), nickel (Ni) and/or copper (Cu) included in the organic solvent purified according to the present disclosure may have a concentration of 5 ppt or less, more preferably 3 ppt or less, and most preferably 2 ppt or less.

Specifically, examples of the impurities to be discharged through the side discharge stream 130 connected to the lower part of the distillation column 120 may include one or more selected from among N-propyl alcohol, 2-pentanone, 4-methyl-2-pentanone, 2-butanol, isopropyl acetate, nonanaldehyde, decanaldehyde, T-amyl alcohol and diacetone alcohol and metal impurities.

The distillation column 120 may be formed as one distillation column as illustrated in FIG. 1, may also be formed with two or more distillation columns placed in parallel in order to increase purity of a final organic solvent product, and, in addition thereto, may be a multi-walled distillation column, a dividing-wall column and the like in which two or more distillations are performed in one column. However, the distillation column 120 is not limited thereto.

The purification process of an organic solvent according to one embodiment of the present disclosure includes a step of (b) discharging a portion of the organic solvent including impurities through the side discharge stream 130 connected to the lower part of the distillation column 120.

The impurities separated in the distillation column 120 may be discharged to the impurity discharge tank 140 through the side discharge stream 130 together with a portion of the organic solvent. In the present disclosure, it is experimentally identified herein that purity of a final purified organic solvent may be improved by discharging a portion of the organic solvent including the impurities through the side discharge stream 130 at a flow rate of 0.5% to 10%, preferably at a flow rate of 1.0% to 10%, with respect to an input flow rate of the organic solvent supplied to the distillation column in the step (a), more preferably an input flow rate of the organic solvent introduced to the raw material tank 110. Specifically, organic impurities such as acetone and/or metal impurities included in a final purified organic solvent may be effectively controlled accordingly. When the discharge flow rate is less than the above-mentioned range, purity is not appropriate even after the purification is completed, and the discharge flow rate being greater than 10% increases the numerical level of the impurities and causes a decrease in the product yield, which reduces an inherent ability of the organic solvent. For example, cleaning performance may be reduced in the case of a cleaning solution, and wafer defects may be caused in the case of a semiconductor cleaning solution.

In addition, performing the discharging of the step (b) as a continuous process has an advantage of being very superior in reducing organic impurities, particularly N-propyl alcohol, compared to a process of periodic discharging.

The organic solvent purification process of the present disclosure may further include a step of liquefying the organic solvent, which is distilled through the distillation column 120 in the step (a), through a condenser (not shown). Two or more of the condensers being installed to be connected in parallel has a process advantage of preventing a loss since the organic solvent that the first condenser fails to condense is re-condensed through the second or higher condenser.

The organic solvent purification process of the present disclosure may further include, after the step (a) and prior to the step (b), a step of re-vaporizing the liquefied impurities discharged through the lower part of the distillation column. Referring to FIG. 1, this is a step performed in a reboiler 121, and since it is difficult to completely separate impurities with one distillation, a portion of the organic solvent including impurities is returned to the distillation column 120, and vaporization and liquefaction are continuously repeated, leading to redistillation. Herein, methods known in the art may be used without limit, however, in one embodiment of the present disclosure, a heat exchange method (thermosyphon reboiler) using a density difference in vapor heat is suitable.

The organic solvent purification process of the present disclosure may further include a step of refluxing the organic solvent distilled in the distillation column 120 through the step (a) in a reflux tank 122. The organic solvent refluxed in the reflux tank 122 may undergo a redistillation process in the distillation column to obtain an organic solvent with higher purity. In addition, the organic solvent that has undergone repeated distillation may be sent to a product tank 150 after inspecting purity in the reflux tank 122, and therefore, a purified organic solvent with higher purity may be obtained.

### <Organic Solvent and Storage Process of Organic Solvent>

The scope of the present disclosure includes an organic solvent purified according to the organic solvent purification process of the present disclosure, and a storage process of the organic solvent. The description provided in the purification process of an organic solvent may be applied to the organic solvent and the storage process of the organic solvent of the present disclosure without limit.

Specifically, the organic solvent purified according to the present disclosure may have a content of at least one of N-propyl alcohol, 2-pentanone, 4-methyl-2-pentanone, 2-butanol, isopropyl acetate, nonanaldehyde, decanaldehyde, T-amyl alcohol and diacetone alcohol of 2 ppm or less and preferably less than 1 ppm in concentration, and may have a content of at least one of iron (Fe), chromium (Cr), nickel (Ni) and/or copper (Cu) of 5 ppt or less, more preferably 3 ppt or less and most preferably 2 ppt or less in concentration.

The organic solvent storage process of the present disclosure may include a step of storing the organic solvent purified according to the process described above at a temperature of lower than 35° C., preferably at a temperature of 30° C. or lower than 30° C.

The organic solvent of the present disclosure may have an acetone content of 2 ppm or less in concentration and preferably less than 1 ppm in concentration after storing for 7 days at a temperature of below zero to lower than 35° C., preferably 30° C. or lower.

Hereinafter, experimental examples including specific examples and comparative examples are presented in order to help understand the present disclosure, however, these are for illustrative purposes only and do not limit the scope of appended claims. It will be obvious to those skilled in the art that various changes and modifications may be made for the examples within the category and the scope of technical ideas of the present disclosure, and it is also reasonable that such changes and modifications fall within the scope of the appended claims.

### EXAMPLE AND COMPARATIVE EXAMPLE

### Example: Continuous Discharge Through Side Discharge Stream

### 1. Effect Depending on Ratio of Continuous Discharge Through Side Discharge Stream

The following Table 1 and Table 2 show, when purifying a raw material (main constituent: isopropyl alcohol) according to an embodiment of the organic solvent purification process of the present disclosure illustrated in FIG. 1, results of measuring the amounts of impurities detected in the final purified organic solvent depending on the amount of discharge in the side discharge stream 130 of the purification tower 100. Specifically, the measurement of each impurity except for the ‘raw material level’ in the following table was conducted in the reflux tank 122 immediately before going to the product tank 150. The ‘raw material level’ is a value obtained by measuring impurities in the raw material before the organic solvent purification of the present disclosure.

### (1) Measurement of Amount of Discharge (%) Through Side Discharge Stream

The amount of discharge (%) in the side discharge stream was measured using the following method. A raw material (main constituent: isopropyl alcohol) was introduced to the raw material tank 110 in FIG. 1 at a flow rate of 4100 kg/hr to 4300 kg/hr (measured through flow meter instrument). After that, a discharge flow rate in the side discharge stream 130 of the purification tower 100 of FIG. 1 was measured in the same manner, and the amount of discharge in the side discharge stream was calculated according to the following equation. The results are shown in the following Table 1 and Table 2.

Amount of discharge through side discharge stream (%)=[Discharge flow rate through side discharge stream 130/Input flow rate to raw material tank 110]*100

### (2) Measurement of Organic Impurities and Metal Impurities

For the final purified organic solvent on which the organic solvent (isopropyl alcohol) purification was performed through the Example, that is, for the organic solvent collected in the reflux tank 122 of FIG. 1, organic impurities were measured using a GC method (Shimadzu Corporation GC-2010 PLUS) and metal impurities were measured using an ICP-MSMS method (Agilent Technologies, Inc. 8900), and the results are shown in Table 1 and Table 2.

TABLE 1

| Amount of | | | | | | | |
|---|---|---|---|---|---|---|---|
| Discharge through | Raw | | | | | | |
| Side Discharge | Material | | | | | | |
| Stream (%) | Level | 0.1 | 0.5 | 1.0 | 2.0 | 3.0 | 4.0 |
| N-propyl Alcohol | 5 | 4 | 2 | <1 | <1 | <1 | <1 |
| (NPA) | | | | | | | |
| 2-Pentanone | <1 | <1 | <1 | <1 | <1 | <1 | <1 |
| Iron (Fe) | 300 | 5~9 | 3~5 | 1~3 | 1~3 | 1~2 | 1~2 |
| Chromium (Cr) | 10 | 5~8 | 3~5 | 1~3 | 1~3 | 1~2 | 1~2 |
| Nickel (Ni) | 250 | 5~7 | 2~5 | 1~2 | 1~2 | 1~2 | 1~2 |
| Copper (Cu) | 10 | 5~7 | 2~5 | 1~2 | 1~2 | 1~2 | 1~2 |

(Unit: ppm, metal (Fe, Cr, Ni, Cu) unit: ppt)

TABLE 2

| | Amount of Discharge through | | | | | |
|---|---|---|---|---|---|---|
| | Side Discharge Stream (%) | | | | | |
| | 5.0 | 6.0 | 7.0 | 8.0 | 9.0 | 10.0 |
| N-propyl Alcohol (NPA) | <1 | <1 | <1 | <1 | <1 | <1 |
| 2-Pentanone | <1 | <1 | <1 | <1 | <1 | <1 |
| Iron (Fe) | 1~2 | 1~2 | 1~2 | 1~2 | 1~2 | 1~2 |
| Chromium (Cr) | 1~2 | 1~2 | 1~2 | 1~2 | 1~2 | 1~2 |
| Nickel (Ni) | 1~2 | 1~2 | 1~2 | 1~2 | 1~2 | 1~2 |
| Copper (Cu) | 1~2 | 1~2 | 1~2 | 1~2 | 1~2 | 1~2 |

(Unit: ppm, metal (Fe, Cr, Ni, Cu) unit: ppt)

According to the experimental data of Table 1 and Table 2, results of less than 1 ppm were obtained in all the organic impurity categories when the amount of discharge in the side discharge stream was 1.0% or greater. In the case of Fe, Cr, Ni and Cu, which are representative metals of stainless steel, among the metal impurities, results of 1 ppt to 2 ppt were obtained in the amount of discharge in the side discharge stream of 30% or less and 1.0% or greater in the purified organic solvent with respect to the impurities included in the supplied organic solvent, indicating that a final purified organic solvent having a favorable quality level was able to be obtained.

On the other hand, in the results of discharge of less than 0.5%, the purity of the organic solvent decreased with NPA (N-propyl alcohol) being greater than 1 ppm, which may resultantly lead to a possibility of defect occurrences in a semiconductor process.

In other words, purity of a final purified organic solvent increases in general when an organic solvent including impurities is discharged through a side discharge stream, however, the effect of significantly improving purity of a purified organic solvent was identified when discharging the solvent including purities through a side discharge stream at a flow rate of preferably 0.5% to 10%, more preferably 1.0% to 10%, with respect to an input flow rate of an organic solvent raw material supplied to a distillation column.

### 2. Relationship Between Amount of Discharge Through Side Discharge Stream and Production Yield

A production yield of the final purified organic solvent depending on the amount of discharge in the side discharge stream (%) measured above is shown in Table 3.

TABLE 3

| Amount of Discharge through Side | Production |
|---|---|
| Discharge Stream (%) | Yield (%) |
| 1 | 99 |
| 2 | 98 |
| 3 | 97 |
| 4 | 96 |
| 5 | 95 |
| 6 | 94 |
| 7 | 93 |
| 8 | 92 |
| 9 | 91 |
| 10 | 90 |
| 11 | 89 |

The production yield is directly related to product quality and manufacturing cost, and therefore, reasonably setting the amount of discharge in the side discharge stream is important. The production yield means a value obtained by subtracting the amount of discharge in the side discharge stream (%) based on a total 100% of the input flow rate of the organic solvent supplied to the entire purification tower, and according to the experimental data of Table 3, the amount of discharge in the side discharge stream being greater than 10% is not suitable since the production yield decreases to less than 90%.

### 3. Amount of Generated Acetone Depending on Storage Temperature of Purified Organic Solvent

The following Table 4 shows results of identifying the amount of detected acetone for each temperature after sealing the organic solvent purified according to an embodiment of the organic solvent purification process of the present disclosure in a 50 ml brown glass bottle, placing the bottle in a device where the temperature is maintained, and storing the bottle for 7 days. ‘Immediately after purification’ is a result of measuring the organic solvent that has undergone the purification process according to an embodiment of the organic solvent purification process of the present disclosure immediately at room temperature (about 25° C.).

TABLE 4

| | Immediately | | | | | | | |
|---|---|---|---|---|---|---|---|---|
| Storage | after | | | | | | | |
| Temperature (° C.) | Purification | −5 | 10 | 20 | 30 | 35 | 40 | 50 |
| Amount of Detected | <1 | <1 | <1 | <1 | <1 | 40 | 60 | 90 |
| Acetone (ppm) | | | | | | | | |

According to the experimental data of Table 4, it was identified that, as a result of the test depending on storage temperature, the datum at room temperature was maintained at the temperature below zero and the temperatures of 30° C. or lower, but the amount of acetone increased from the moment the temperature exceeded 35° C. In addition, estimating that the acetone amount was higher at 40° C. and 50° C. compared to at 35° C., it may be seen that the energy of high temperature facilitates acetone regeneration. In other words, it was identified that the organic solvent purified according to the present disclosure was suitable when stored at a temperature of lower than 35° C., preferably 30° C. or lower.

4. Amount of Acetone when Long-Term Storing Purified Organic Solvent at 40° C.

The amount of acetone was long-term evaluated at 40° C., which exceeds the appropriate temperature condition, to identify the trend, and the results are shown the following Table 5. After filling an ISO tank lorry with the organic solvent purified according to an embodiment of the organic solvent purification process of the present disclosure, the organic solvent was long-term stored while maintaining a temperature of 40° C. to conduct the evaluation. The organic solvent was collected in a 50 ml brown glass bottle, and the bottle was sealed and then moved to an analysis room for analysis. As an environmental condition of the analysis room, a temperature of 15° C. to 30° C. and a humidity of 30% to 60% are suitable. ‘Same-day measurement’ is a result of measuring the organic solvent that has undergone the purification process immediately.

TABLE 5

| Category | Acetone (ppm) |
|---|---|
| Same-day Measurement | <1 |
| Day 7 | 60.2 |
| Day 14 | 60.5 |
| Day 21 | 61.3 |
| Day 28 | 60.0 |

According to the experimental data of Table 5, it may be identified that the temporarily increased acetone amount tends to be maintained. In other words, since the amount of regenerated acetone for each temperature is fixed at a certain amount, it may be said that long-term storage does not significantly affect the amount of generated acetone. For example, according to the experimental data, the amount of generated acetone at 40° C. is about 60 ppm.

### Comparative Example: Periodic Discharge Through Side Discharge Stream

Purification of an organic solvent was performed in the same manner as in Example except that, in the organic solvent purification process of the present disclosure, periodic discharge was conducted in cycles of 12 hours, 1 day and 3 days instead of the continuous discharge in the side discharge stream.

For the final purified organic solvent that has undergone the organic solvent (isopropyl alcohol) purification through side discharge for each cycle, the amounts of NPA (N-propyl alcohol), a representative high-boiling point impurity, and acetone, a representative low-boiling point impurity, in the organic solvent were measured for each time period shown in the following Table 6, and the results are shown in the following Table 6.

TABLE 6

| | 12 Hours | | 1 Day | | 3 Days | |
|---|---|---|---|---|---|---|
| Category | NPA | Acetone | NPA | Acetone | NPA | Acetone |
| Raw Material | 4 | <1 | 4 | <1 | 5 | <1 |
| Level | | | | | | |
| 3 hr | 1 | 2 | 2 | 2 | 1 | 2 |
| 6 hr | 2 | 4 | 3 | 3 | 2 | 7 |
| 12 hr | 4 | 9 | 5 | 10 | 5 | 11 |
| 18 hr | — | — | 6 | 15 | 6 | 17 |
| 24 hr | — | — | 8 | 20 | 7 | 22 |
| 32 hr | — | — | — | — | 8 | 30 |
| 48 hr | — | — | — | — | 10 | 38 |
| 60 hr | — | — | — | — | 12 | 45 |
| 72 hr | — | — | — | — | 13 | 50 |

(unit: ppm)

According to the experimental data of Table 6, a modest effect was obtained for NPA (N-propyl alcohol) at an early stage of the periodic discharge, however, the amount of generated acetone increased and continuously increased as the cycle is extended since it is a low boiling-point impurity. In other words, the biggest problem of periodic discharge is that acetone increases by heat, and the concentrated amount increases exponentially. Accordingly, it was identified that, when performing a process according to the organic solvent purification process of the present disclosure, continuous and frequent discharge is more suitable for improving purity of a final product.

### REFERENCE NUMERAL

100: Purification Tower
110: Raw Material Tank
111: Dry Bed Unit
120: Distillation Column
121: Reboiler
122: Reflux Tank
130: Side Discharge Stream
140: Impurity Discharge Tank
150: Product Tank

1. A purification process of an organic solvent, the process comprising:
(a) performing distillation by supplying an organic solvent to a distillation column; and
(b) discharging a portion of the organic solvent including impurities through a side discharge stream connected to a lower part of the distillation column,
wherein the discharging of (b) discharges a portion of the organic solvent including impurities at a flow rate of 0.5% to 10% with respect to an input flow rate of the organic solvent supplied to the distillation column in (a).

2. The process of claim 1, wherein the organic solvent is an alcohol-based organic solvent.

3. The process of claim 2, wherein the organic solvent is one or more selected from among isopropyl alcohol, propyl alcohol, butyl alcohol and hexyl alcohol.

4. The process of claim 1, wherein the impurity is a substance having the same or higher boiling point compared to the organic solvent.

5. The process of claim 4, wherein the impurity includes one or more selected from among N-propyl alcohol, 2-pentanone, 4-methyl-2-pentanone, 2-butanol, isopropyl acetate, nonanaldehyde, decanaldehyde, T-amyl alcohol, diacetone alcohol and metal impurities.

6. The process of claim 1, wherein the discharging of (b) discharges a portion of the organic solvent including impurities at a flow rate of 1.0% to 10% with respect to an input flow rate of the organic solvent supplied to the distillation column in (a).

7. The process of claim 1, wherein the discharging of (b) is performed as a continuous process.

8. The process of claim 1, which is for reducing a content of at least one of iron (Fe), chromium (Cr), nickel (Ni) and copper (Cu) included in the organic solvent to 30% or less.

9. The process of claim 1, further comprising storing the purified organic solvent at a temperature of lower than 35° C.

10. The process of claim 9, further comprising storing the purified organic solvent at a temperature of 30° C. or lower.

11. The process of claim 1, further comprising at least one of removing moisture included in the organic solvent prior to (a);
re-vaporizing the liquefied impurities discharged through the lower part of the distillation column after (a) and prior to (b);
liquefying the distilled organic solvent through a condenser after (a); and
refluxing the organic solvent distilled in the distillation column after (a).

12. An organic solvent in which a content of at least one of N-propyl alcohol, 2-pentanone, 4-methyl-2-pentanone, 2-butanol, isopropyl acetate, nonanaldehyde, decanaldehyde, T-amyl alcohol and diacetone alcohol is 2 ppm or less in concentration, and a content of at least one of iron (Fc), chromium (Cr), nickel (Ni) and copper (Cu) is 5 ppt or less in concentration.
